(19) United States
(12) Patent Application Publication
(10) Pub. No.: US 2013/0071464 A1
(43) Pub. Date: Mar. 21, 2013

(54) COMPOSITION COMPRISING PHOSPHONIC ACID POLYMER FOR USE AS TISSUE ENGINEERING FIBROUS SCAFFOLD

(71) Applicants: Sandra Downes, York (GB); Moshen Zakikhani, Birmingham (GB); Anita Kaur Bassi, Derby (GB); Julie Elizabeth Gough, Bury (GB)

(72) Inventors: Sandra Downes, York (GB); Moshen Zakikhani, Birmingham (GB); Anita Kaur Bassi, Derby (GB); Julie Elizabeth Gough, Bury (GB)

(73) Assignee: THE UNIVERSITY OF MANCHESTER, Manchester (GB)

(21) Appl. No.: 13/390,383

(22) PCT Filed: Aug. 6, 2010
(86) PCT No.: PCT/GB2010/001482

§ 371 Date: Sep. 26, 2012

(30) Foreign Application Priority Data
Aug. 14, 2009 (GB) 0914200.1

(51) Int. Cl. A61L 27/18; C09D 185/02; C08F 230/02; A61K 31/78

(52) U.S. Cl. CPC A61L 27/18; A61K 31/78; C09D 185/02; C08F 230/02
USPC 424/443; 424/400; 424/78.31; 524/547; 526/278

(57) ABSTRACT

A fibrous tissue scaffold wherein fibres of the scaffold comprise a phosphonic acid polymer. The fibres preferably incorporate a vinylphosphonic acid—acrylic acid copolymer, and may further include polycaprolactone. There is further described medical implant coating comprising a phosphonic acid polymer, a biocompatible fibre comprising a phosphonic acid polymer and a biocompatible polymer comprising polycaprolactone and a phosphonic acid polymer.

The present invention relates to biocompatible materials, such as tissue scaffolds in the form of beads, films and the like, coatings for medical implants, compositions and formulations.

Biocompatible materials, such as bone graft substitutes, produced by synthetic means are becoming ever more important as new products and methods are sort to treat medical conditions resulting from experiences ranging from acute trauma (e.g. fractures of the bone or tooth due to accident) to chronic illness (e.g. osteoporosis, Paget's disease, or tooth decay).

In healthy individuals relatively minor fractures to bone resulting from acute trauma usually heal naturally over a short period of time. In more severe cases, a bone graft or implant of some kind may be required. There can be long term clinical problems if the bone defects are exceptionally large. In osteoporotic patients, which is a specific bone condition, the risk of initial fracture is greatly increased as compared to a healthy individual and the ability for the individual to recover naturally is significantly reduced. There is a clinical need for a product(s) as bone graft substitutes which could be used clinically for patients with osteoporosis who suffer a fracture and/or require a bone filler in conjunction with a joint replacement.

Osteoporosis is a disease of the bone in which bone mineral density is lower than normal and the microarchitecture and protein structure of the bone is disrupted as a result of homeostatic imbalance between bone formation by cells known as osteoblasts and bone resorption by cells known as osteoclasts.

Osteoporosis is a chronic illness that affects an estimated 75 million people worldwide and there were an estimated 9 million osteoporotic fractures in 2000. Twenty-four percent of women and 33% of men suffering from osteoporosis die within one year of suffering a hip fracture. As Europe's population ages, the number of people affected by osteoporosis is set to rise significantly; indications suggest that hip fractures alone are expected to double in the next 50 years. The total direct cost of osteoporosis in Europe is estimated at £21 billion and is expected to increase to £51 billion by 2050.

Paget's disease (osteitis deformans) is a chronic illness which is similar to osteoporosis in so far as it also results from an imbalance between bone formation and bone resorption. As a result, there are similarities between the treatments administered to patients suffering from the two conditions.

Currently, osteoporotic patients and Paget's disease patients are administered a group of drugs called bisphosphonates which are taken orally. Bisphosphonates act by binding to hydroxyapatite in bone; consequently the drug is internalised by osteoclasts and this leads to osteoclast apoptosis. Unfortunately, patients being administered bisphosphonates can suffer from very undesirable side-effects, exhibiting, for example, fever and flu like symptoms. There is also no commercially available implant designed for patients suffering from osteoporosis and/or Paget's disease, or that can be implanted at a specific site where it is needed. Bone graft substitutes for both normal and osteoporotic patients are currently human/animal derived or synthetic scaffolds however they lack bioactivity and osteoinductivity.

An object of the present invention is to obviate or mitigate one or more of the aforementioned problems and/or to provide improved biocompatible materials, such as bone graft substitutes, designed for tissue regeneration and/or repair.

According to a first aspect of the present invention there is provided a fibrous tissue scaffold comprising a phosphonic acid polymer.

The first aspect of the present invention relates to a scaffold (i.e. a two or three dimensional supporting structure) for tissue attachment and/or growth in which the scaffold structure comprises a phosphonic acid polymer. The phosphonic acid polymer may be incorporated into the material of the fibres of the scaffold and/or provided as a coating over at least a region of the scaffold. Thus, some or all of the fibres of the scaffold may be formed from a phosphonic acid polymer and/or some or all of the fibres of the scaffold may be provided with a coating containing a phosphonic acid polymer. By way of example, fibres of the scaffold may be formed from one or more types of phosphonic acid polymer, and/or the coating may incorporate one or more types of phosphonic acid polymer. Moreover, the fibres may be formed from one type of phosphonic acid polymer and the coating may contain a different type of phosphonic acid polymer. In addition, phosphonic acid moieties may be formed by means of chemical reaction on the surface of the scaffold or by functionalisation of the scaffold with an appropriate molecule containing phosphonic acid moieties.

Reference herein to a “phosphonic acid polymer” encompasses any type of polymer produced by polymerisation of a monomer incorporating a phosphonic acid group and a polymerisable moiety (e.g. a vinyl group), optionally in combination with one or more other types of monomer. It will also be appreciated that reference herein to a “phosphonic acid polymer” also encompasses polymers of phosphonic acid salts. Moreover, the aforementioned polymers may comprise at least one phosphono or phosphino moiety.

Since the scaffold comprises a phosphonic acid polymer with phosphorus as a pendant group on the backbone, which is analogous to the active moiety in bisphosphonates, the scaffold possesses the ability to bind to the predominant constituent of bone and teeth, hydroxyapatite, and to be internalised by resorbing osteoclasts. Chemical analysis of a scaffold in accordance with the first aspect of the present invention, described in more detail in the Examples, has indicated that calcium is concentrated in areas where there is an increased phosphorous concentration suggesting that there is binding between phosphorous and calcium. In vitro and in vivo studies have demonstrated cellular interactions with the scaffold leading to cell attachment and proliferation and the formation of extracellular matrix. Preferred embodiments of the scaffold possess a porous structure which allows the migration of cells throughout and therefore further increases the area accessible for cellular interactions.

In light of the above, the tissue engineered scaffold of the first aspect of the present invention is therefore eminently suitable for use by patients suffering from osteoporosis, Paget's disease and/or chronic dental illness to improve bone mass and/or bone stock at areas where it is most needed. It is also anticipated that since the phosphonic acid polymer-containing scaffold contains only the active moiety of the bisphosphonates family of compounds (i.e. the phosphorous-carbon bond), the scaffold of the first aspect of the present invention can be considered as a non-pharmaceutical product and should eliminate some, if not all, of the unwanted side effects currently associated with conventional bisphosphonate treatment.

A still further advantage of the scaffold of the first aspect of the present invention is that in preferred embodiments it is biodegradable therefore eliminating the need for further invasive surgery consequently reducing hospital expenses and risk of infection.

A yet further advantage arises due to the fact that various embodiments of the scaffold and constituent fibres are hydrophilic which greatly assists cell adhesion as compared to more hydrophobic materials. There is also likely to be a chemical reaction between the biopolymer and hydroxyapatite found in the bone matrix. This will further improve bone repair and healing.

As will be appreciated from the discussion below relating to the fabrication of the scaffold another advantage of the present invention is that the method by which the scaffold can be produced is relatively simple and does not require the use of, for example, UV sensitive monomers or zwitterionic compounds.

The skilled person will understand that the scaffold is suitable for wider application in the repair and regeneration of bone and teeth in otherwise healthy individuals. For example, in the treatment of individuals who have suffered some form of acute trauma to bone or tooth.

A first preferred embodiment of the present invention provides a fibrous tissue scaffold wherein fibres of the scaffold are formed from a phosphonic acid polymer.

The first preferred embodiment thus relates to a scaffold in which the material from which the structure is produced incorporates a phosphonic acid polymer. That is, the phosphonic acid polymer forms an integral part of the scaffold material as distinct from having been applied in some form of surface treatment process to provide a phosphonic acid surface coating to the scaffold. It will be appreciated, however, that the scaffold can be provided with any desirable type of surface coating, including a coating incorporating a phosphonic acid-based compound or polymer to modify or enhance the chemical, biological and/or physical properties of the scaffold incorporating phosphonic acid polymer-based fibres.

Moreover, the scaffold of the first preferred embodiment of the present invention may incorporate fibres made exclusively from a phosphonic acid polymer or the scaffold may incorporate a combination of different types of fibres wherein some, but not all, of the fibres making up the scaffold are formed from a phosphonic acid polymer.

A second preferred embodiment provides a fibrous tissue scaffold wherein fibres of the scaffold are provided with a coating containing a phosphonic acid polymer.

The phosphonic acid polymer-containing coating may be applied to substantially all of the fibres making up the scaffold or just a portion thereof. Moreover, the or each fibre of the scaffold provided with such a coating may be substantially completely covered with the coating or the coating may be applied to just a portion or region of the or each fibre. The coating may cover from around 0.00001 to 100% of the fibre's surface area, more preferably around 0.001 to 100% of the fibre's surface area, and still more preferably around 0.1 to 100% of the fibre's surface area. Moreover, the coating may be one layer thick or may be up to thousands of layers thick depending upon the intended application of the coated fibres.

Regardless of whether the phosphonic acid polymer is provided within the structure of the scaffold fibres as in the first preferred embodiment, or is provided as a coating as in the second embodiment, or a combination of both, it is preferred that the total concentration of the phosphonic acid polymer is greater than around 7 to 8 w/v %, and/or preferably does not exceed around 20 w/v %. A preferred range for the concentration of the phosphonic acid polymer is around 7 to 15 w/v %. More preferably the phosphonic acid polymer concentration is around 10 to 18 w/v %, still more preferably around 13 to 17 w/v %, and most preferably around 15 w/v %.

The starting material that is formed into the fibres of the scaffold according to the first preferred embodiment of the present invention may be a phosphonic acid oligomer, homopolymer or heteropolymer (e.g. copolymer, terpolymer, etc), or may be a mixture or blend of any such phosphonic acid moieties in combination with one or more other polymers. The molecular weight of the polymer could be that of the combination of a few monomer units forming the polymer (as defined by the regulatory bodies as “polymer”) to several million depending upon the intended application of the polymer. More preferably the molecular weight is in the range of around 100 g/mol to 500,000 g/mol, and most preferably the molecular weight is in the range of around 100 g/mol to 200,000 g/mol.

In a preferred embodiment the fibres of the scaffold are formed from a homopolymer of a phosphonic acid monomer, such as vinyl phosphonic acid (VPA). Alternatively, the fibres of the scaffold may be formed from a co-polymer of a phosphonic acid monomer copolymerised with a second type of polymerisable monomer. Preferred copolymerisable monomers include polymerisable carboxylic acid monomers, such as acrylic acid and methacrylic acid (referred to herein generically as “(meth)acrylic acid”) monomers.

In accordance with preferred embodiments of the present invention, the polymer may incorporate an active moiety selected from a phosphono-component and/or phosphino-component. The phosphono-component or phosphino-component may consist essentially of vinylphosphonic acid (VPA), vinylidene-1,1-diphosphonic acid (VDPA), a phosphono substituted mono- or di-carboxylic acid, hypophosphorus acid or a salt, such as an alkali metal salt of hypophosphorus acid. By way of example, the phosphono-component may consist essentially of a homopolymer of VPA, VDPA, or phosphono-succinic acid.

A composition incorporating the polymer may further include one or more additional components, such as unsaturated sulphonic acid, saturated or unsaturated carboxylic acids, unsaturated amides, primary or secondary amines, polyalkylene imines, or amine-terminated polyalkylene glycols. For example, the polymeric composition may consist essentially of a copolymer of vinylphosphonic acid (VPA) with vinylsulphonic acid (VSA), or with acrylic acid (AA), methacrylic acid (MAA) or acrylamide. Alternatively, the polymeric composition may consist essentially of a copolymer of VDPA with VSA, or with AA, MAA or acrylamide. As another example, the polymeric composition may consist essentially of a terpolymer of VDPA, VSA and either AA, MMA or acrylamide. Alternatively, the polymeric composition may consist essentially of the reaction product VPA, or VDPA, or a mixture of VPA and VDPA, and any one of the following:
a. a primary amine;
b. a secondary amine;
c. a polyethylene imine;
d. an amine terminated polyethylene or polypropylene glycol (e.g. jeffamines); and/or
e. a hypophosphorus acid or salt thereof.

A particularly preferred copolymer for use in fabricating fibres for the scaffold is poly (vinyl phosphonic acid-co-acrylic acid).

It will be appreciated that any of the aforementioned polymers, copolymers or terpolymers may comprise at least one phosphono or phosphino moiety.

The scaffold according to the first aspect of the present invention is preferably formed from one or more biocompatible polymers. Preferably fibres of the scaffold are made from or comprise polycaprolactone. Polycaprolactone (poly-ε-caprolactone) is a biocompatible material which is approved by the US Food and Drug Administration (FDA) for use in biomedical applications. It is thus eminently suitable for application in tissue scaffolds according to the first aspect of the present invention. In accordance with the present invention, other biocompatible polymers can be used in addition to, or in place of, polycaprolactone, such as poly(glycolic acid), poly(lactic acid), poly(lactic-co-glycolic acid), and/or poly(methyl methacrylate).

In a particularly preferred embodiment of the first aspect of the present invention there is provided a fibrous tissue scaffold wherein at least some of the fibres are formed from a polymer solution comprising polycaprolactone and poly (vinyl phosphonic acid-co-acrylic acid).

The scaffold according to the first aspect of the present invention preferably comprises sub-micron diameter fibres. The fibres may possess an average diameter in the range of around 10 to 1000 nm, more preferably around 100 to 500 nm, and yet more preferably around 200 to 400 nm. It is particularly preferred that the diameters possess an average diameter of around 250 to 300 nm.

The fibres within the scaffold may have a substantially ordered arrangement, partly ordered arrangement or be essentially randomly arranged. It is particularly preferred that the fibres are randomly arranged since this most closely mirrors the physical structure of the natural biological environment in which the scaffold is intended to be employed.

The scaffold may be provided in the form of a three dimensional bead or packing material of any desirable size and shape. The scaffold material may be utilised in a specific application in the form of beads which have a single size and shape, or which have a range of sizes and/or shapes. It is envisaged that it will be preferable to use beads of the phosphonic acid polymer material having a range of different sizes from relatively small to relatively large so that the beads can self-organise themselves so as to adopt the most appropriate packing arrangement within a particular target site.

Alternatively, the scaffold may be provided as an essentially two dimensional sheet or strip of fibrous material. It will, of course, be appreciated that even such “two dimensional” materials will possess a third dimension, e.g. thickness, but that materials of this kind possess two predominant dimensions, e.g. width and length.

In a preferred embodiment the scaffold according to the first aspect of the present invention is provided in the form of a polymeric packing material for insertion into a bone or tooth cavity. It will be appreciated that it is important that the packing materials is capable of withstanding the levels of compressive forces experienced by the implant site within the body. While the packing material may be of any desirable size and/or shape, it is preferred that the packing material is in the form of a pellet. The pellets used in a particular application may all be of the same size and shape, or may be of a range of sizes and/or shapes to enable them to assume the optimal packing arrangement for the size and shape of the target site. The porosity and micro/macro structures can be altered to provide the optimum structures for specific chemical applications.

In a further preferred embodiment the scaffold is provided in the form of a polymeric sheet, which can exhibit any desirable degree of flexibility to suit an intended site of implantation.

The scaffold is preferably porous, which is advantageous since it allows infiltration of cells throughout the structure of the scaffold and affords a significantly increased specific surface area for cell growth as compared to non-porous materials. Porous scaffolds also facilitate the penetration of nutrients from cell culture media through the scaffold to the growing cells supported by the scaffold. Additionally, providing the scaffold with a porous structure affords a means to manipulate and control various physical, chemical and biological properties of the scaffold so that they can be tailored to suit a particular application.

A particularly preferred method for producing the phosphonic acid-based fibres in the scaffold of the present invention is electrospinning a solution of a suitable phosphonic acid polymer, or polymer mixture or blend containing a suitable phosphonic acid polymer.

During the electrospinning process a polymer is fed through a needle at a constant rate and a voltage is applied. The voltage leads to the formation of a Taylor's cone at the needle tip and when the electrostatic forces overcome the surface tension of the polymer solution, a polymer jet is formed which is attracted to a grounded collector plate.

The feed rate of the polymer may be altered to control the nature of the fibres formed and to take account of the properties of the particular polymer being spun. It is preferred that feed rate is around 0.01 to 0.1 ml/min, more preferably around 0.05 ml/min. The applied voltage may be varied according to the properties of the particular polymer being electrospun and/or the desired properties of the final fibres. The applied voltage may be around 5 to 40 kV, more preferably around 10 to 30 kV and is most preferably around 20 kV. The separation of the needle from the collector plate is a further parameter which can be changed to control the nature of the final fibres depending upon the polymer being used. The needle may be spaced around 10 to 20 cm from the collector plate, and is preferably spaced around 15 cm from the collector plate.

In order to fabricate a scaffold comprised primarily of well-defined fibres, rather than non-fibrous agglomerations of polymer, it is preferred that the electrospinning solution should contain at least around 6 w/v % of the polymer or polymer mixture being spun to form the fibres (which may or may not contain phosphonic acid polymer), more preferably around 7 to 15 w/v %of the polymer or polymer mixture, and most preferably around 10 w/v % of the polymer or polymer mixture.

The polymer solution being electrospun may incorporate any appropriate solvent. A preferred solvent is acetone.

As described above, a preferred embodiment of the fibrous scaffold according to the first aspect of the present invention contains fibres made from a polymer mixture containing a vinyl phosphonic acid/acrylic acid copolymer and polycaprolactone. In this case, it is preferred that the following electrospinning parameters are adopted: 10 w/v % polymer mixture in acetone (VPA concentration around 15 w/v %); applied voltage of 20 kV; polymer flow rate of 0.05 ml/min; and a needle / collector distance of 15 cm.

A second aspect of the present invention provides a biocompatible fibre comprising a phosphonic acid polymer.

The biocompatible fibre according to the second aspect of the present invention may incorporate a phosphonic acid polymer as a coating and/or within the structure of the fibre as described above in relation to the first aspect of the present invention. The fibre according to the second aspect may therefore possess any of the physical, chemical and/or biological properties set out above in respect of the phosphonic acid polymer-containing fibres employed in the scaffold according to the first aspect of the present invention.

A third aspect of the present invention provides a medical implant coating comprising a phosphonic acid polymer.

A fourth aspect of the present invention provides medical implant provided with a coating comprising a phosphonic acid polymer.

The coating of the third and fourth aspects may comprise one or more types of phosphonic acid polymer, optionally in combination with one or more further polymers. The coating may be a phosphonic acid homopolymer or heteropolymer, or may be a mixture or blend of any such phosphonic acid polymer in combination with one or more other polymers. The molecular weight of the polymer could be that of the combination of a few monomer units forming the polymer (as defined by the regulatory bodies as “polymer”) to several million depending upon the intended application of the polymer. More preferably the molecular weight is in the range of around 100 g/mol to 500,000 g/mol, and most preferably the molecular weight is in the range of around 100 g/mol to 200,000 g/mol.

In a preferred embodiment the coating comprises a homopolymer of a phosphonic acid monomer, such as vinyl phosphonic acid (VPA). In another embodiment, the coating is a co-polymer of a phosphonic acid monomer copolymerised with a second type of polymerisable monomer, such as a polymerisable carboxylic acid monomer. Preferred examples of copolymerisable monomers include (meth)acrylic acid monomers.

In accordance with preferred embodiments of the present invention, the polymer may incorporate an active moiety selected from a phosphono-component and/or phosphino-component. The phosphono-component or phosphino-component may consist essentially of vinylphosphonic acid (VPA), vinylidene-1,1-diphosphonic acid (VDPA), a phosphono substituted mono- or di-carboxylic acid, hypophosphorus acid or a salt, such as an alkali metal salt of hypophosphorus acid. By way of example, the phosphono-component may consist essentially of a homopolymer of VPA, VDPA, or phosphono-succinic acid.

A composition incorporating the polymer may further include one or more additional components, such as unsaturated sulphonic acid, saturated or unsaturated carboxylic acids, unsaturated amides, primary or secondary amines, polyalkylene imines, or amine-terminated polyalkylene glycols. For example, the polymeric composition may consist essentially of a copolymer of vinylphosphonic acid (VPA) with vinylsulphonic acid (VSA), or with acrylic acid (AA), methacrylic acid (MAA) or acrylamide. Alternatively, the polymeric composition may consist essentially of a copolymer of VDPA with VSA, or with AA, MAA or acrylamide. As another example, the polymeric composition may consist essentially of a terpolymer of VDPA, VSA and either AA, MMA or acrylamide. Alternatively, the polymeric composition may consist essentially of the reaction product VPA, or VDPA, or a mixture of VPA and VDPA, and any one of the following:
a. a primary amine;
b. a secondary amine;
c. a polyethylene imine;
d. an amine terminated polyethylene or polypropylene glycol (e.g. jeffamines); and/or
e. a hypophosphorus acid or salt thereof.

A particularly preferred copolymer for use in the coating is poly (vinyl phosphonic acid-co-acrylic acid).

The coating preferably comprises one or more biocompatible polymers in addition to the phosphonic acid polymer(s), such as polycaprolactone (poly-ε-caprolactone). In accordance with the present invention, other biocompatible polymers can be used such as poly(glycolic acid), poly(lactic acid), poly(lactic-co-glycolic acid), poly(methyl methacrylate).

In a particularly preferred embodiment of the third and/or fourth aspects of the present invention there is provided a medical implant coating comprising polycaprolactone and poly (vinyl phosphonic acid-co-acrylic acid).

It is envisaged that the coating may be applied to any desirable medical implant. The coating is perceived as being particularly suitable for use in applications in which a surface of the medical implant will contact adjacent areas of bone and/or tooth. It is therefore preferred that the surface(s) of the medical implant to which the coating is intended to be applied or has been applied is that surface or are those surfaces which will contact bone and/or tooth following implantation. Medical implant devices may be fabricated from a range of different biocompatible materials, a common one of which is titanium. Example 8 below describes the successful application of a polycaprolactone / poly(vinyl phosphonic acid-co-acrylic acid) polymer coating to a titanium substrate in which after only three days significant levels of osteoblast attachment were observed thereby demonstrating the suitability of this coating for application as a medical coating at the implant/bone interface of the implant.

A fifth aspect of the present invention provides biocompatible polymer composition comprising polycaprolactone and a phosphonic acid polymer.

The polymer composition may be a mixture, blend or heteropolymer incorporating copolymerised phosphonic acid and caprolactone monomers, optionally including one or more further monomers. Where the polymer composition is a mixture or blend of a phosphonic acid component and a polycaprolactone component, the phosphonic acid component may be a phosphonic acid homopolymer or heteropolymer. The molecular weight of the polymer could be that of the combination of a few monomer units forming the polymer (as defined by the regulatory bodies as “polymer”) to several million depending upon the intended application of the polymer. More preferably the molecular weight is in the range of around 100 g/mol to 500,000 g/mol, and most preferably the molecular weight is in the range of around 100 g/mol to 200,000 g/mol.

In a preferred embodiment the phosphonic acid component of the polymer composition comprises a homopolymer of a phosphonic acid monomer, such as vinyl phosphonic acid (VPA). In another embodiment, the phosphonic acid component is a co-polymer of a phosphonic acid monomer copolymerised with a second type of polymerisable monomer, such as a polymerisable carboxylic acid monomer, for example, a (meth)acrylic acid monomer.

In accordance with preferred embodiments of the present invention, the polymer may incorporate an active moiety selected from a phosphono-component and/or phosphino-component. The phosphono-component or phosphino-component may consist essentially of vinylphosphonic acid (VPA), vinylidene-1,1-diphosphonic acid (VDPA), a phosphono substituted mono- or di-carboxylic acid, hypophosphorus acid or a salt, such as an alkali metal salt of hypophosphorus acid. By way of example, the phosphono-component may consist essentially of a homopolymer of VPA, VDPA, or phosphono-succinic acid.

A composition incorporating the polymer may further include one or more additional components, such as unsaturated sulphonic acid, saturated or unsaturated carboxylic acids, unsaturated amides, primary or secondary amines, polyalkylene imines, or amine-terminated polyalkylene glycols. For example, the polymeric composition may consist essentially of a copolymer of vinylphosphonic acid (VPA) with vinylsulphonic acid (VSA), or with acrylic acid (AA), methacrylic acid (MAA) or acrylamide. Alternatively, the polymeric composition may consist essentially of a copolymer of VDPA with VSA, or with AA, MAA or acrylamide. As another example, the polymeric composition may consist essentially of a terpolymer of VDPA, VSA and either AA, MMA or acrylamide. Alternatively, the polymeric composition may consist essentially of the reaction product VPA, or VDPA, or a mixture of VPA and VDPA, and any one of the following:
a. a primary amine;
b. a secondary amine;
c. a polyethylene imine;
d. an amine terminated polyethylene or polypropylene glycol (e.g. jeffamines); and/or
e. a hypophosphorus acid or salt thereof.

A particularly preferred copolymer for use in the phosphonic acid component of the polymer composition is poly (vinyl phosphonic acid-co-acrylic acid). Thus, a particularly preferred embodiment of the fifth aspect of the present invention provides a polymer composition comprising a mixture of polycaprolactone and poly (vinyl phosphonic acid-co-acrylic acid).

Polymer compositions according to the fifth aspect of the present invention are eminently suitable for application as coatings on medical implants as mentioned above in respect of the third and fourth aspects of the present invention. Moreover, polymer compositions according to the fifth aspect of the present invention are eminently suitable for incorporation into formulations suitable for injection. Such formulations may be provided as liquid pharmaceutical compositions in the form of sterile solutions or suspensions for administration by injection. It will be appreciated that injectable compositions in accordance with the invention may also include binders, suspending agents and preservatives as necessary. Suitable examples of such agents will be well known to those skilled in the art.

A sixth aspect of the present invention provides a method of culturing mammalian tissue cells, the method comprising culturing mammalian tissue cells on a fibrous tissue scaffold, fibres of the scaffold being comprising a phosphonic acid polymer, and exposing said cells to a cell culture medium to facilitate adherence of said cells to the scaffold.

A seventh aspect of the present invention provides a method of repairing or regenerating mammalian tissue comprising culturing mammalian tissue cells on a fibrous tissue scaffold, fibres of the scaffold comprising a phosphonic acid polymer, exposing said cells to a cell culture medium to facilitate adherence of said cells to the scaffold, and placing the scaffold with cells adhered thereto adjacent to the mammalian tissue in need of repair or regeneration.

An eighth aspect of the present invention provides a method of treating osteoporosis comprising administering to an individual suffering from osteoporosis a therapeutic amount of a composition comprising a phosphonic acid polymer.

A ninth aspect of the present invention provides a method of treating Paget's disease comprising administering to an individual suffering from Paget's disease a therapeutic amount of a composition comprising a phosphonic acid polymer.

A tenth aspect of the present invention provides a method of treating an acute trauma to a bone site comprising administering to said site in an individual having suffered said acute trauma a therapeutic amount of a composition comprising a phosphonic acid polymer.

In the eighth, ninth and tenth aspects of the present invention the composition is preferably in accordance with the above-defined fifth aspect of the present invention. Moreover, the composition is preferably administered directly to the site or sites within the subject which have been affected by osteoporosis, Paget's disease or an acute trauma (e.g. a broken bone or tooth). Administration may be by way of implantation of a scaffold according to the first aspect of the present invention, e.g. as a fibrous sheet or film, or as a pellet, bead or packing material, or by way of injection.

An eleventh aspect of the present invention relates to an injectable medicament for use in the treatment of osteoporosis comprising a phosphonic acid polymer.

A twelfth aspect of the present invention relates to an injectable medicament for use in the treatment of Paget's disease comprising a phosphonic acid polymer.

A thirteenth aspect of the present invention relates to an injectable medicament for use in the treatment of an acute trauma to a bone site comprising a phosphonic acid polymer.

The medicament employed in the eleventh, twelfth and thirteenth aspects of the present invention is preferably a composition in accordance with the above-defined fifth aspect of the present invention.

As the skilled person will appreciate a therapeutically effective amount of the above-mentioned compositions relates to an amount sufficient to treat the condition or disease being treated.

The subject being treated may be human or animal. The bone being treated may be any bone forming part of the skeleton of the subject or may be a tooth.

Compositions in accordance with the invention suitable for localised parenteral administration may be prepared by mixing a phosphonic acid polymer with optional physiologically acceptable carriers, excipients or stabilizers in the form of for example lyophilised and non-lyophilised powder formulations for reconstitution prior to use, non-aqueous and aqueous solutions, and semi-solid formulations. Acceptable carriers, including excipients, are non-toxic to recipients at the dosages and concentrations employed, and include, but are not limited to, appropriate buffers, antioxidants, toxicity modifiers, preservatives and/or anionic surfactants.

Composition in accordance with the present invention may be presented in the form of a vial, an ampoule, or a pre-filled syringe of, either a sterile solution, a sterile suspension or any other pharmaceutically acceptable form of presentation suited to localised parenteral delivery.

It will be appreciated that a scaffold employed in the sixth to tenth aspects of the present invention may comprise any of the features set out in the preferred embodiments of the scaffold according to the first aspect of the present invention. Moreover, a scaffold employed in the sixth to tenth aspects of the present invention may incorporate biocompatible fibres according to the second aspect of the invention.

Preferred embodiments of aspects of the present invention will now be demonstrated with reference to the following non-limiting examples, in which:

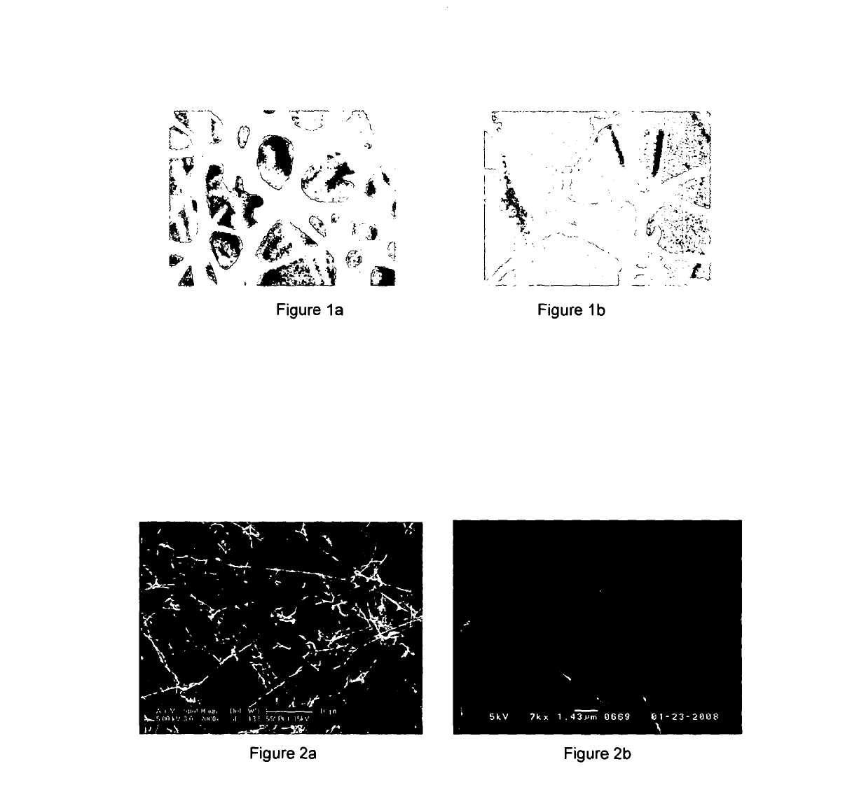

FIGS. 1a and 1b are scanning electron microscope (SEM) images of healthy bone and osteoporotic bone respectively;

FIGS. 2a and 2b are scanning electron microscope (SEM) images of fibrous materials formed by electrospinning polymer solutions containing 5% and 10% polycaprolactone (PCL) respectively;

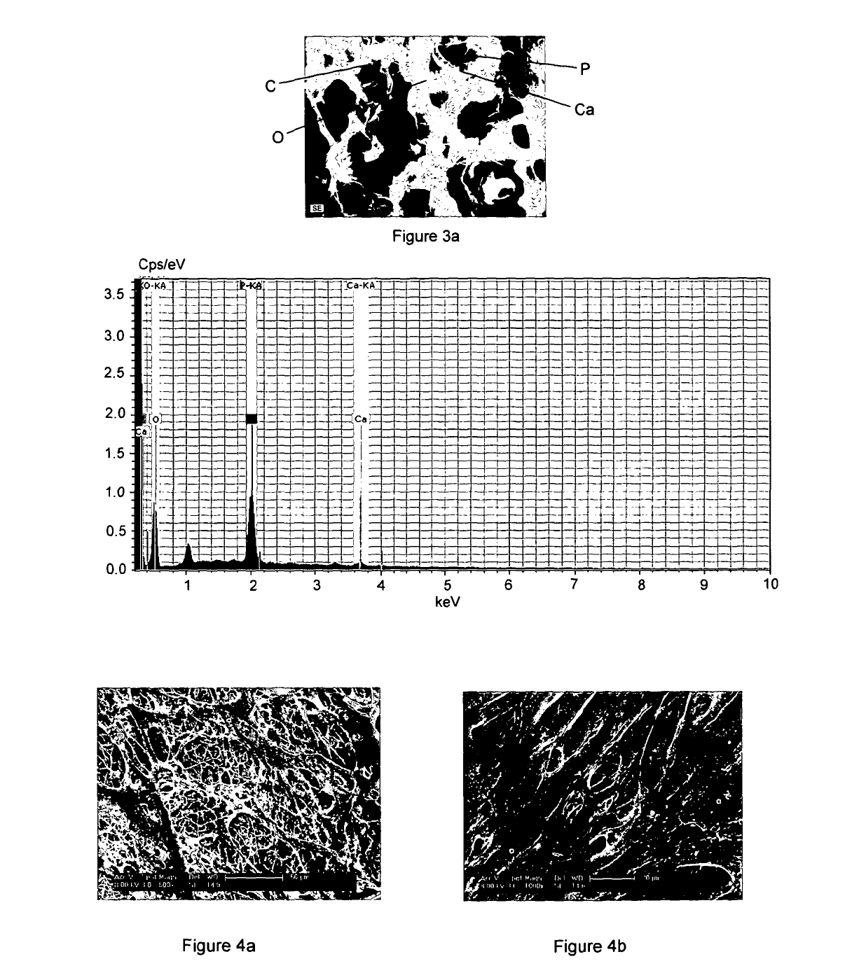

FIG. 3a is a colour-coded image of the internal structure of a tissue scaffold according to the first aspect of the present invention generated from energy dispersive x-ray (EDX) analysis of the scaffold

FIG. 3b is an energy dispersive x-ray (EDX) spectrum for the material shown in FIG. 3a;

FIGS. 4a and 4b are scanning electron microscope (SEM) images at 500× and 1000× magnification respectively of human osteoblasts adhered to a tissue scaffold according to the first aspect of the present invention;

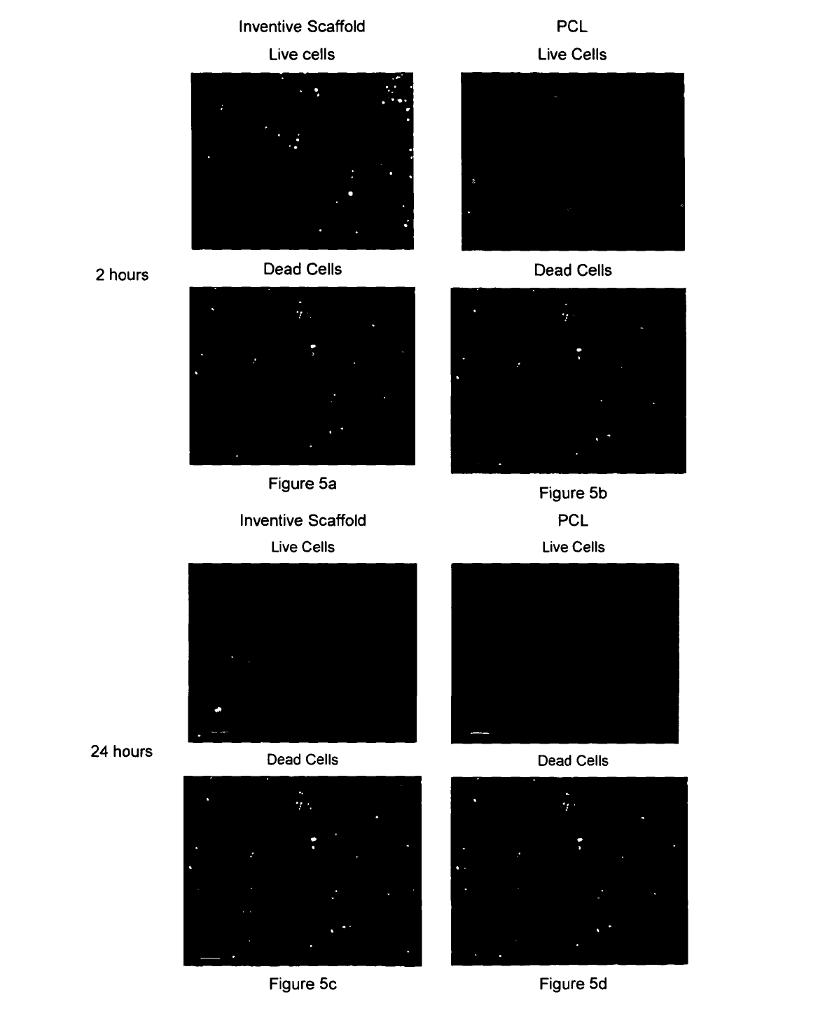

FIGS. 5a to 5d are pairs of images of live (upper) and dead (lower) cells adhered to a tissue scaffold according to the first aspect of the present invention (FIGS. 5a and 5c) and a tissue scaffold made from polycaprolactone (PCL) acting as a control (FIGS. 5b and 5d). FIGS. 5a and 5b are images taken after 2 hrs and FIGS. 5c and 5d are images taken after 24 hours;

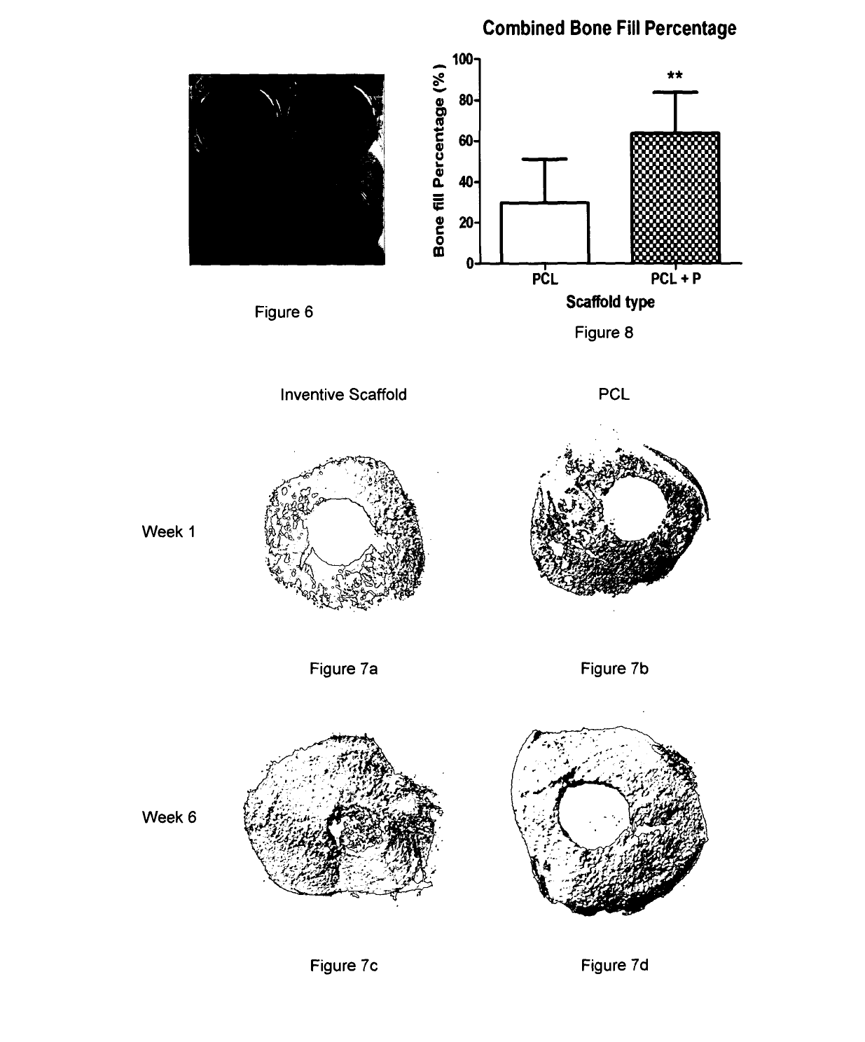

FIG. 6 is a photograph of tissue scaffolds analysed using Alizarin red staining to determine levels of calcium ion binding to each scaffold. The tissue scaffolds on the left were made from PCL to act as a control and the tissue scaffolds on right were in accordance with the first aspect of the present invention;

FIGS. 7a to 7d are microscopic x-ray computed tomography (micro-CT) images of calvarial samples provided with critical sized (1.5 mm diameter) defects and subsequently treated with a tissue scaffold according to the first aspect of the present invention (FIGS. 7a and 7c) or a control tissue scaffold made from polycaprolactone (PCL) (FIGS. 7b and 7d). FIGS. 7a and 7b are images taken after one week and FIGS. 7c and 7d are images taken after 6 weeks;

FIG. 8 is a graph showing the level of bone formation observed for the tissue scaffold according to the first aspect of the present invention illustrated in FIG. 7c and the control tissue scaffold made from PCL illustrated in FIG. 7d;

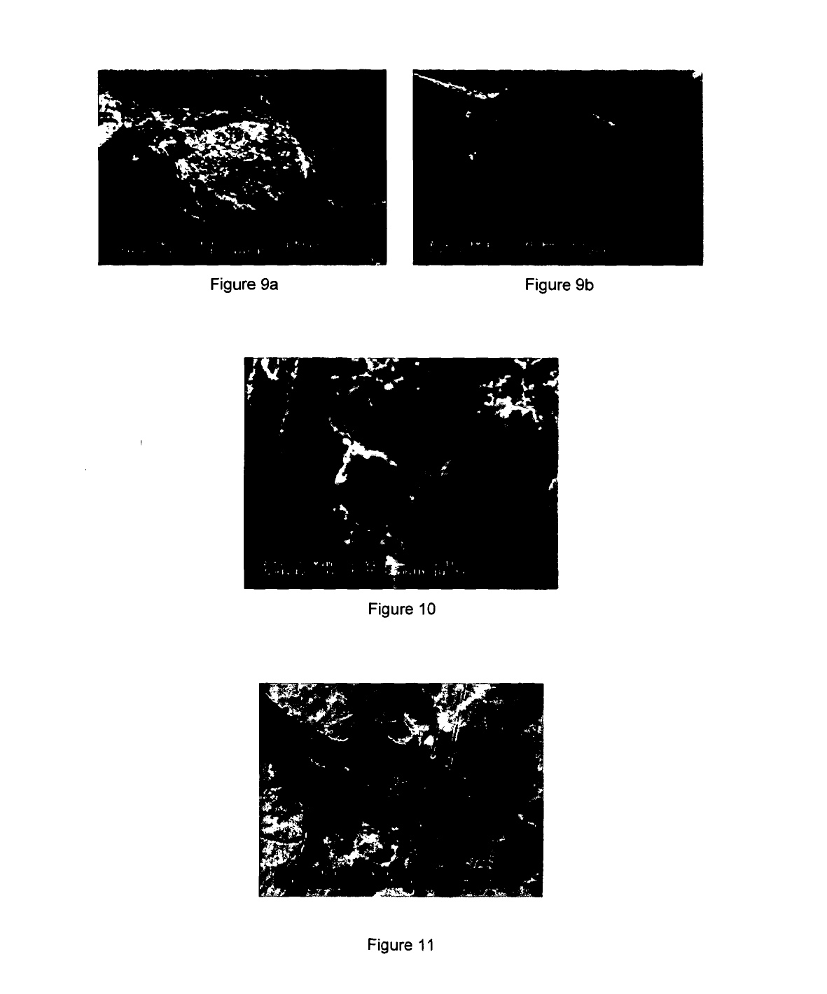

FIGS. 9a and 9b are scanning electron microscopy (SEM) images of calvarial samples provided with critical sized (1.5 mm diameter) defects and subsequently treated with a control tissue scaffold made from polycaprolactone (PCL) (FIG. 9a) and a tissue scaffold according to the first aspect of the present invention (FIG. 9b). Images taken 6 weeks after implantation;

FIG. 10 is a scanning electron microscopy (SEM) image of a scaffold according to the first aspect of the present invention after implantation showing hydroxyapatite-like formations; and

FIG. 11 is a scanning electron microscopy (SEM) image of a surface of a titanium substrate coated with a polymeric coating according to the second aspect of the present invention showing osteoblast attachment and formation;

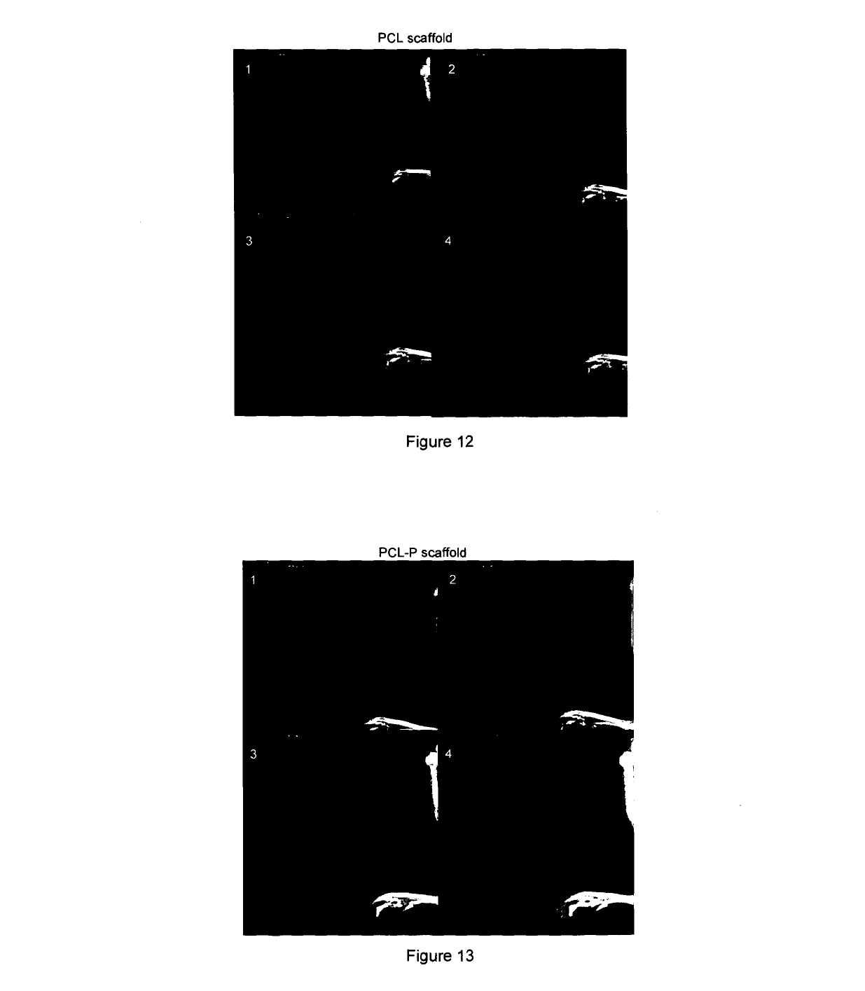

FIG. 12 is a Faxitron X-ray image of an in vivo subcutaneous implant made of fibres of PCL without any further derivitisation to act as a control;

FIG. 13 is a Faxitron X-ray image of an in vivo subcutaneous implant made of fibres of PCL coated with a phosphonic acid polymer composition to provide a scaffold according to the present invention;

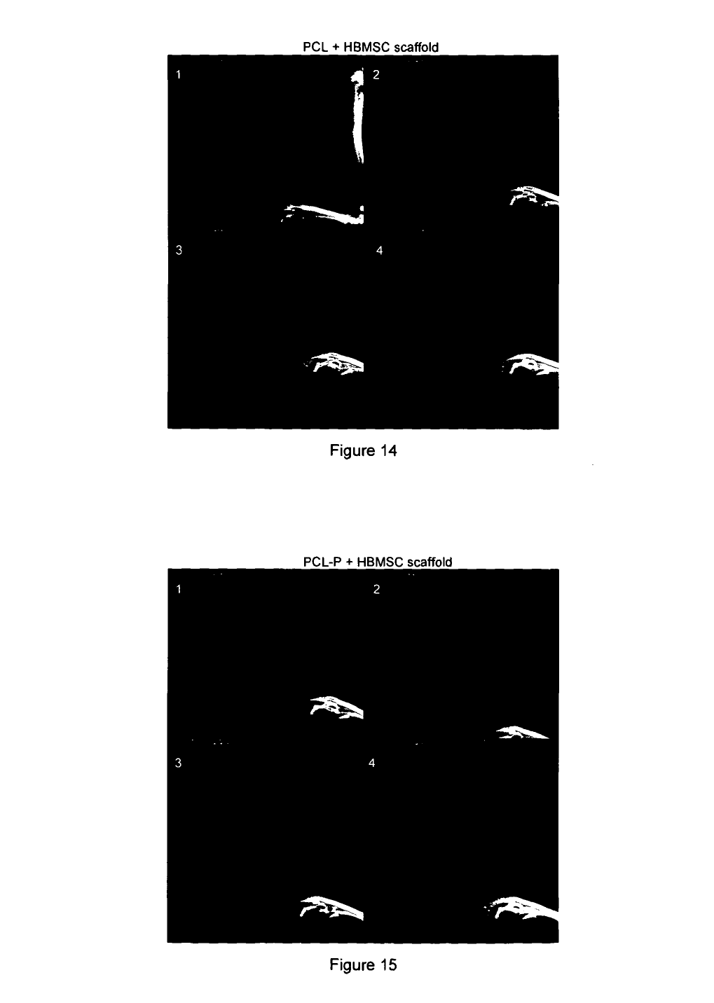

FIG. 14 is a Faxitron X-ray image of an in vivo subcutaneous implant made of fibres of PCL without any further derivitisation which has been immersed in human bone marrow stromal cells (HBMSC) to act as a second control;

FIG. 15 is a Faxitron X-ray image of an in vivo subcutaneous implant made of fibres of PCL coated with a phosphonic acid polymer composition and immersed in human bone marrow stromal cells (HBMSC) to provide a scaffold according to the present invention;

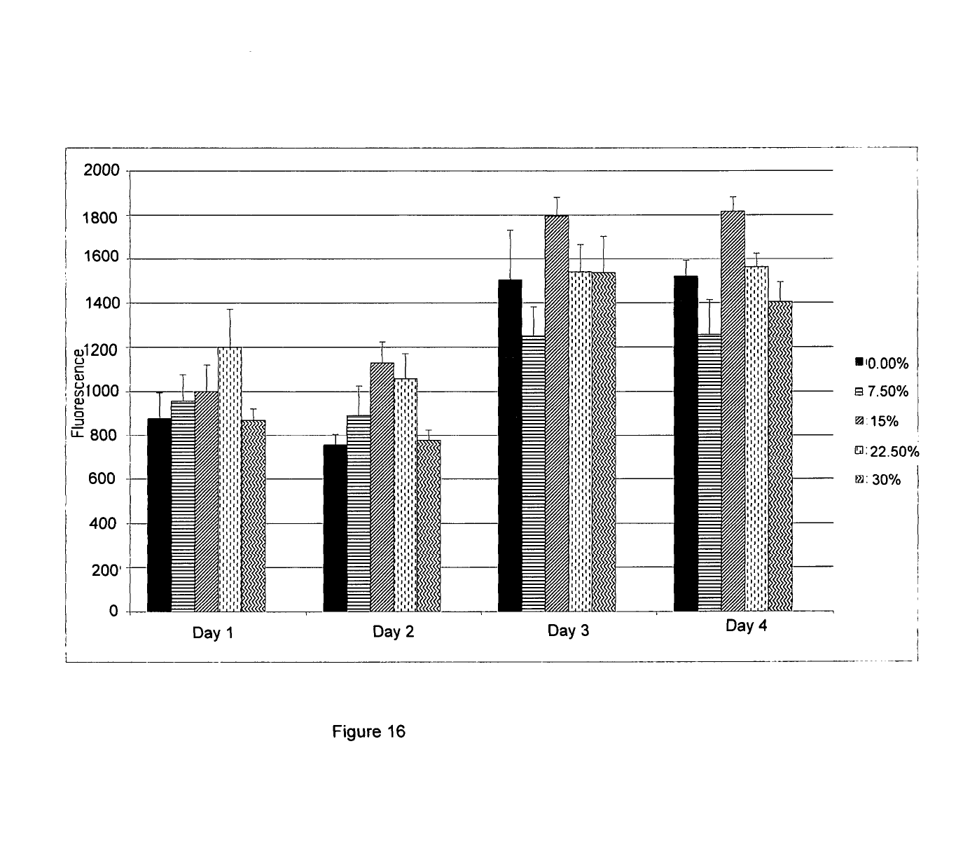

FIG. 16 is a graph of fluorescence intensity illustrating the effects of different phosphonic acid polymer concentrations on the response of osteoblasts;

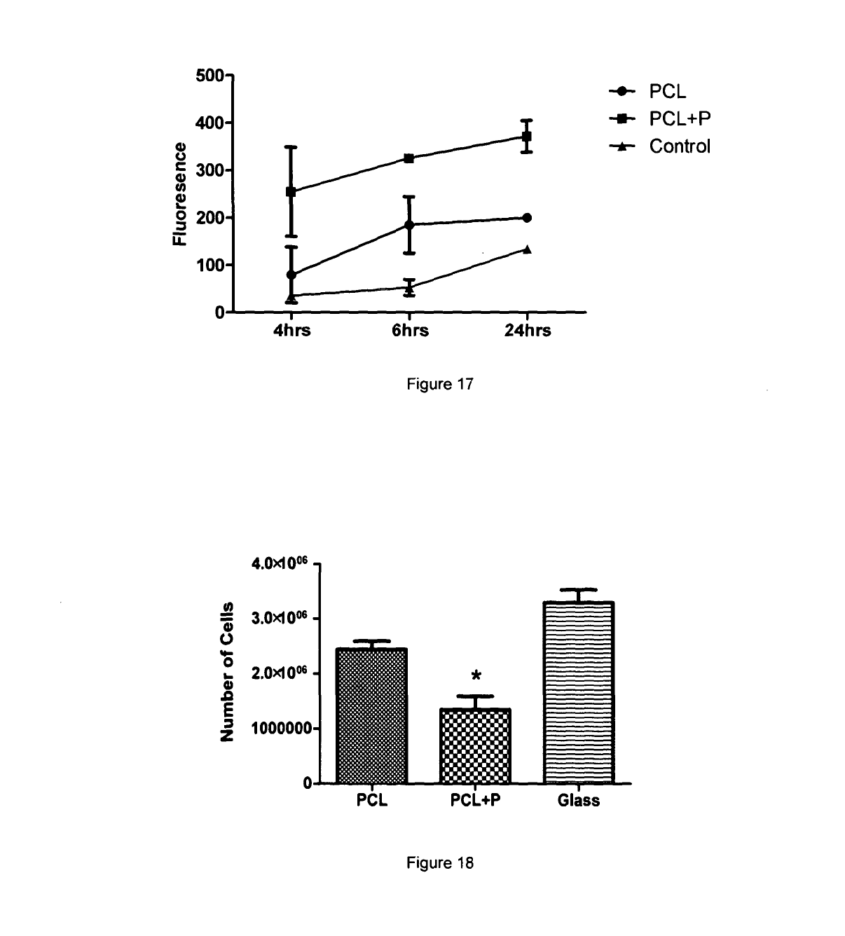

FIG. 17 is a graph of osteoclast apoptosis measured using the Apopercentage kit from Biocolor;

FIG. 18 is a graph of cell count for three scaffolds, one scaffold according to the present invention (PCL+P) and two scaffolds for comparison (Control and PCL); and

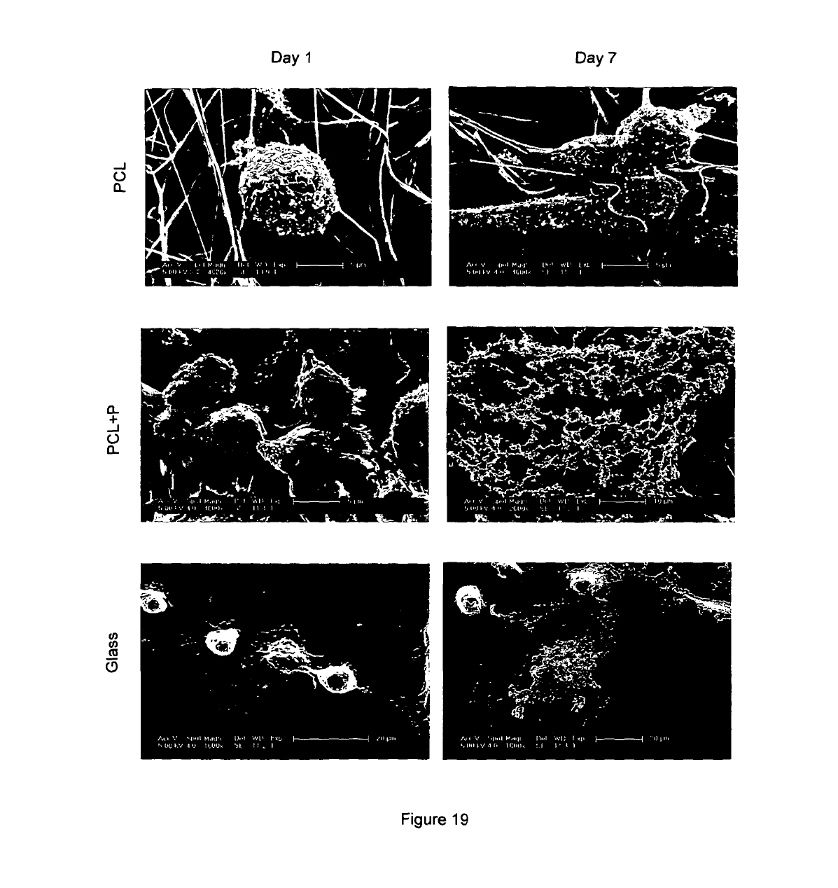

FIG. 19 shows scanning electron microscopy images of cellular material attached to three scaffolds, one scaffold according to the present invention (PCL+P) and two scaffolds for comparison (Control and PCL).

### EXAMPLES

### Example 1

### Optimisation of Scaffold Fabrication

Various electrospinning parameters were investigated to determine which would be best for preparing fibrous scaffolds which can be coated with a phosphonic acid polymer composition and/or contain fibres made from a phosphonic acid-containing polymer composition. Different concentrations of polycaprolactone were used varying from 2% to 20%, and the voltages applied were varied to achieve optimum conditions.

Polycaprolactone fibres were fabricated by an electrospinning process. Acetone was used as a solvent to dissolve polycaprolactone pellets under gentle stirring and heat to obtain a polycaprolactone concentration of 10 w/v %.

The polymer solution was placed in a 10 ml syringe fitted to a blunted needle with a diameter of 0.8 mm. A high voltage power supply was attached to the needle and a voltage of 20 kV was applied. A grounded collector plate was located at a fixed distance of 15 cm from the needle tip.

A syringe pump was used to feed the polycaprolactone polymer through the needle at a constant rate of 0.05 ml/min. A taylor cone was formed when the voltage was applied and this led to the formation of a polymer jet which was attracted to the grounded collector plate.

Scanning electron microscopy (SEM) analysis demonstrated that lower PCL concentrations of 2 w/v % and 5 w/v % gave rise to beads within the fibrous mat (see FIG. 2a) whereas a higher PCL concentration, for example a PCL concentration of 10 w/v %, yielded clearly defined fibres with an average fibre diameter of 266 nm (see FIG. 2b). Beaded fibrous structures are undesirable for the aforementioned application. Concentrations of PCL much higher than 10 w/v %, for example, above 15 w/v % are not amenable to electrospinning due to the viscosity of their solutions.

Fibrous tissue scaffolds according to the present invention for use in the following Examples were then prepared by electrospinning a 10 w/v % PCL polymer solution as described above and then immersing the PCL scaffold in a 15 w/v % polyvinyl phosphonic acid polymer aqueous solution containing deionised water for 24 hours. The resulting PCL/PVPA film was air dried for 48 hours and then heated to 55 ° C. for 24 hours.

### Example 2

### Calcium Ion Adsorption to Scaffold

Scaffolds according to the first aspect of the present invention containing phosphonic acid polymer were immersed in a calcium rich solution for three days. After 3 days, energy dispersive x-ray (EDX) analysis demonstrated an increase in calcium ions at areas of high phosphorous concentration (see FIG. 3a). Calcium phosphate formation is vital for bone formation and in vitro aggregation of the two elements is promising for in vivo bone formation. The presence of phosphorous and calcium is confirmed in the EDX spectrum as shown in FIG. 3b. The spectrum also confirms that phosphorous was still present after 3 days of immersion in a liquid.

### Example 3

### Cell Growth on Scaffold

Human osteoblasts have been shown to attach and proliferate on scaffolds according to the first aspect of the present invention as shown in FIG. 4. The cells were observed to have spread over the surface of the scaffolds and displayed normal osteoblast structure.

### Example 4

### Cell Survival on Scaffold

Live/dead staining distinguishes live cells (visible in each upper image in FIG. 5) from dead cells (visible in each lower image in FIG. 5). After 2 hours, cells on both a control scaffold (pure PCL) and a scaffold according to the first aspect of the present invention containing phosphonic acid polymer are rounded as would be expected. There appeared to be more live cells on the scaffold containing the phosphonic acid polymer as shown in FIG. 5. On the pure PCL scaffold there were fewer live cells, and more dead cells. After 24 hours the cells were observed to have spread across the surfaces of the scaffolds and, as with the 2 hour time point, there were fewer dead cells on the scaffold containing the phosphonic acid polymer according to the first aspect of the present invention.

### Example 5

Mineralising Capacity of Scaffold—Alizarin Staining Human osteoblasts were seeded on to two control scaffolds (pure PCL) and two scaffolds according to the first aspect of the present invention. Alizarin red staining to identify the presence of calcium ions was carried out after 3 weeks. It was observed that the scaffolds containing the phosphonic acid polymer were stained a deep red colour, whereas the pure PCL scaffolds only had specks of red as shown in FIG. 6. This result suggests that calcium apatite had formed much more rapidly on the scaffolds containing the phosphonic acid polymer according to the first aspect of the present invention.

### Example 6

### Mineralising Capacity of Scaffold—Bone Mineral Volume

The calvarial from 4 day old mice were extracted aseptically and a 1.5 mm diameter critical sized defect was created, which under normal circumstances would not heal. A scaffold containing phosphonic acid polymer according to the first aspect of the present invention was added to one such defect and a control scaffold (pure PCL) added to another defect. A total of five pairs of samples were prepared in this way for testing. After 6 weeks of culture (osteogenic media—DMEM supplemented with 10° A) FBS, 100 μg/ml penicillin/streptomycin, 0.05 mM ascorbic acid, 0.1 μM dexamethasone, 10 mM β-glycerophosphate disodium salt) there was a significant difference in bone mineral volume between the two samples when examined using microscopic x-ray computed tomography (micro-CT) as shown in FIG. 7. Scanning electron microscopy (SEM) images of the treated samples also demonstrated that the scaffold according to the first aspect of the present invention displayed better integration into the defect site than the control scaffold (see FIGS. 9a and 9b).

As very few osteoclasts are present in the calvarial we can assume that the phosphonic acid polymer has an effect on osteoblast activity as well as osteoclast activity. The statistical difference in bone fill percentage over the five pairs of samples is shown in FIG. 8 from which it can be concluded that the scaffolds according to the first aspect of the present invention yielded significantly increased bone formation as compared to the control scaffolds.

The suitability of the scaffold according to the first aspect of the present invention for use in biomedical applications was further demonstrated by the observation of hydroxyapatite-like formations following culturing.

### Example 7

### Preparation of Coating Formulation

A solution of poly (vinyl phosphonic acid-co-acrylic acid) [Poly(VPA-co-AA)] containing 10-15% solid polymer in distilled water was prepared.

### Example 8

### Application of Coating Formulation to Substrate

A titanium (Ti) substrate was heated to 100 to 150° C. before being immersed in a pre-heated bath of poly(VPA-co-AA). The Ti substrate was then removed from the bath and washed with water. As a result, a surface of the titanium substrate was coated with a polymeric coating of poly(VPA-co-AA) according to a preferred embodiment of the second aspect of the present invention.

After three days, significant osteoblast attachment to the coating was observed as shown in FIG. 11.

### Example 9

### Subcutaneous Implant Mouse Models

PCL and PCL-P scaffolds were sterilized using an antimycotic/antibiotic (AM/AB) solution (Sigma, UK). 5× AM/AB in 1× PBS for 1 hr; 1× AM/AB in 1× PBS overnight and finally washed in 1× PBS for 24 hrs.

A first set of four PCL scaffolds prepared as described above was tested as described below without further modification (results shown in FIG. 12). A first set of four PCL-P scaffolds according to the present invention prepared as described above was tested as described below without further modification (results shown in FIG. 13).

A second set of four PCL scaffolds prepared as described above was incubated in 1 ml 10% fetal calf serum (FCS) alpha minimal essential medium (MEM) and 1×10⁶ human bone marrow stromal cells (HBMSC) using a rotomix overnight in an incubator at 37° C., 5% CO₂ balanced air before being tested as described below (results shown in FIG. 14).

A second set of four PCL-P scaffolds to the present invention prepared as described above was incubated in 1 ml 10% fetal calf serum (FCS) alpha minimal essential medium (MEM) and 1×10⁶ human bone marrow stromal cells (HBMSC) using a rotomix overnight in an incubator at 37° C., 5% CO₂ balanced air before being tested as described below (results shown in FIG. 15).

For all in vivo studies, female MF-1 nu/nu immunodeficient mice were purchased from Harlan, Loughborough, UK and acclimatized for a minimum of 1 week prior to experimentation. Animals had ad libitum access to standard mouse chow and water at all times, and all procedures were performed with prior received ethical approval and carried out in accordance with the regulations as laid down in the Animals (Scientific Procedures) Act 1986.

In a Class 1 cabinet, mice were weighed and anaesthetized by intraperitoneal injection of fentanyl-fluanisone (Hypnorm) (Janssen-Cilag Ltd) and midazolam (Hypnovel) (Roche Ltd) in sterile water at a ratio of 1:1 and a dose of 10 ml kg⁻¹. A longitudinal incision was made using a No 10 scalpel on one side of the vertebral column. Lateral to the incision a subcutaneous pouch was created using blunt dissection. The scaffolds were subcutaneously placed in the pouches and the incisions were sutured using Michel staple clips. n=4 scaffolds/group were placed in the individual pouches.

The duration of the experiment was 28 days.

At the end of the experiment mice were exposed to rising levels of CO₂, followed by translocation of the upper cervical vertebrae. The subcutaneous cell/scaffold implants were extracted and processed for Faxitron X-ray images to assess if mineralisation had occurred.

With reference to FIGS. 12 to 15, it was observed that the PCL scaffolds that contained no phosphonic acid polymer (FIGS. 12 and 14) showed some evidence of mineralisation but that the level of mineralisation was far lower than could be observed on scaffolds according to the present invention that contained the phosphonic acid polymer (FIGS. 13 and 15).

These results support the in vitro cell culture results described above where there was an increase in mineralisation in the presence of the phosphonic acid polymer. The increase in mineralisation exhibited by scaffolds according to the present invention suggests that the phosphonic acid moiety in the scaffold is enhancing the mineralising ability of the osteoblasts. It should also be noted that in the present Example even though the scaffolds were implanted in a ‘non-bone’ area, calcium apatite formation was observed, which suggests that the scaffold according to the present invention is osteoinductive.

### Example 10

### Osteoblast Culture

Human osteoblast cells (HOBs) were purchased from The European Collection of Cell Cultures, UK. Cells were cultured in Dulbecco's modified Eagles medium (DMEM) (Gibco, UK) supplemented with 10% fetal bovine serum (FBS) (Gibco, UK), antibiotics (100 U/ml penicillin, 100 mg/ml streptomycin) (Gibco, UK), 100 nM dexamethasone (Sigma, UK), 50 uM ascorbic acid (Sigma, UK) and 10 mM β-glycerophosphate (Merck Biosciences, UK).

Cells were cultured in 75 cm² tissue culture flasks; medium was replenished every 2 days. When required cells were detached from the culture flasks by trypsinisation; cell number and viability were checked with trypan blue.

Phosphonic acid-containing scaffolds according to the present invention were immobilised in 24 well plates using CellCrown inserts (Scaffdex, Finland) and sterilised using a series of increasing ethanol concentrations in a laminar flow cabinet.

Following the final immersion in ethanol, the scaffolds were washed with sterile phosphate buffered saline (Gibco, UK) three times to remove any traces of ethanol. Cells were counted and seeded on to the scaffolds at a density of 40,000 cells/cm². Plates were cultured in standard conditions, at 37° C. with 95% humidity and 5% CO₂ for up to 4 days.

### Osteoblast Metabolic Activity

At days 1, 2, 3, and 4, culture medium was removed and samples were washed with sterile phosphate buffered saline, 1 ml of fresh culture medium was added along with 100 μl of AlamarBlue solution (5 mg resazurin salt/40 ml PBS)(Sigma, UK).

The plates were incubated at 37° C. with 95% humidity and 5% CO₂ for 2 hours. After 2 hours, 200 μl of suspension was transferred to a 96 well plate. The fluorescence was measured at an excitation of 530 nm and emission at 590 nm using a flourometer. The results are shown in FIG. 16.

Over the 4-day culture period there was a general increase in fluorescence over time, which suggests an increase in metabolic activity. Over the 4 days, metabolic activity was highest for samples that contained 15% phosphonic acid polymer. There was no significant difference in metabolic activity at day 4 for samples containing 22.5% and 30% phosphonic acid polymer, although these results are lower than that obtained for 15% phosphonate at day 4.

At this stage these initial results suggest that a concentration of the phosphonic acid polymer of around 15% is the optimum level for enhanced osteoblast metabolic activity using the AlamarBlue assay. These results also suggest that as the phosphonic acid polymer concentration is increased above around 20%, the metabolic activity of the cells may be reduced, hinting that too high a concentration of the phosphonic acid polymer may force cells into a dormant state or towards apoptosis.

### Example 11

### Osteoclast Culture

Human osteoclast pre-cursor cells were purchased from Lonza, UK. Cells were seeded on to three scaffolds at a density of 30,000 cells/well in osteoclast basal medium supplemented with RANK-L, M-CSF, FBS, glutamine and antibiotics. The three scaffolds used were a glass scaffold (Control), a scaffold made from PCL (PCL) and a scaffold according to the present invention made from PCL with a coating of phosphonic acid polymer (PCL+P).

At 4 hrs, 6 hrs and 24 hrs samples were tested using the apopercentage apoptosis detection kit (Biocolor, UK). The results are presented in FIG. 17. As can be seen, there was a significant increase in apoptosis on the scaffold according to the present invention (PCL+P) as compared to the other two scaffolds (Control and PCL). An increase in apoptosis suggests an uptake of P-C by the osteoclasts which leads to cell apoptosis.

At day 14, cells were detached from the sample surface and cells were counted using a haemocytometer. The results are presented in FIG. 18. After 14 days in culture there was a significant decrease in cell number on the scaffold according to the present invention (PCL+P) as compared to the other two scaffolds (Control and PCL).

At days 1 and 7, cells were visualised using scanning electron microscopy. The images obtained are presented in FIG. 18. At day 1 the osteoclast pre-cursors have attached to all three types of scaffold, however, at day 7 there is evidence of cell debris on the scaffold according to the present invention (PCL+P) suggesting that the cells have undergone apoptosis. At day 7 on the other two scaffolds (Control and PCL) there is formation of multinucleated cells.

1-36. (canceled)

37. A medical implant coating comprising a phosphonic acid polymer.

38. A coating according to claim 37, wherein said phosphonic acid polymer comprises at least one phosphono or phosphino moiety.

39. A coating according to claim 37, wherein said phosphonic acid polymer is a phosphonic acid homopolymer, or a phosphonic acid heteropolymer incorporating one or more other types of polymer.

40. A coating according to claim 37, wherein said phosphonic acid polymer is a phosphonic acid homopolymer selected from the group consisting of vinyl phosphonic acid, vinylidene-1,1-diphosphonic acid, phosphono-substituted mono-carboxylic acid, phosphono-substituted di-carboxylic acid, hypophosphorus acid, a hypophosphorus acid salt, and phosphono-succinic acid.

41. A coating according to claim 37, wherein said phosphonic acid polymer is a phosphonic acid heteropolymer of phosphonic acid and one or more further monomers selected from the group consisting of carboxylic acid, sulphonic acid, amides, amines, polyalkylene imines, and amine-terminated polyalkylene glycols.

42. A coating according to claim 37 wherein said phosphonic acid polymer is a phosphonic acid heteropolymer selected from the group consisting of vinylphosphonic acid and vinylsulphonic acid, vinylphosphonic acid and acrylic acid, vinylphosphonic acid and methacrylic acid, vinylphosphonic acid and acrylamide, vinylidene-1,1-diphosphonic acid and vinylsulphonic acid, vinylidene-1,1-diphosphonic acid and acrylic acid, vinylidene-1,1-diphosphonic acid and methacrylic acid, vinylidene-1,1-diphosphonic acid and acrylamide, vinylidene-1,1-diphosphonic acid, vinylsulphonic acid and acrylic acid, vinylidene-1,1-diphosphonic acid, vinylsulphonic acid and methacrylic acid, and vinylidene-1,1-diphosphonic acid, vinylsulphonic acid and acrylamide.

43. A coating according to claim 37, wherein said phosphonic acid polymer is poly(vinyl phosphonic acid-co-acrylic acid).

44. A coating according to claim 37, wherein the molecular weight of the phosphonic acid polymer is in the range of around 100 g/mol to 500,000 g/mol.

45. A coating according to claim 37, wherein the coating comprises around 7 to 20 w/v % of the phosphonic acid polymer.

46. A coating according to claim 37, wherein the coating comprises around 15 w/v % of the phosphonic acid polymer.

47. A medical implant provided with a coating comprising a phosphonic acid polymer.

48. An implant according to claim 47, wherein the coating comprises a phosphonic acid polymer.

49. A biocompatible fibre comprising at least one type of phosphonic acid polymer.

50. A fibre according to claim 49, wherein said fibres are formed from a first type of phosphonic acid polymer.

51. (canceled)

52. A fibre according to claim 49, wherein fibres of the scaffold are provided with a coating containing a second type of phosphonic acid polymer.

53. A biocompatible polymer composition comprising polycaprolactone and a phosphonic acid polymer.

54. (canceled)

55. A composition according to claim 53, wherein said composition contains around 7 to 20 w/v % of the phosphonic acid polymer.

56. A composition according to claim 53, wherein said composition contains around 7 to 15 w/v % of the phosphonic acid polymer.

57. (canceled)

58. An injectable medicament for use in the treatment of a condition selected from the group consisting of osteoporosis, Paget's disease and an acute trauma to a bone site comprising a phosphonic acid polymer.

59-62. (canceled)

63. A method of treating a condition selected from the group consisting of osteoporosis, Paget's disease and an acute trauma to a bone site comprising administering to an individual suffering from said condition a therapeutic amount of a composition comprising a phosphonic acid polymer.

64-65. (canceled)
